(19) United States
(12) Patent Application Publication
(10) Pub. No.: US 2019/0105534 A1
(43) Pub. Date: Apr. 11, 2019

(54) APPARATUS FOR SEMI-OCCLUDED VOCAL TRACT EXERCISES

(71) Applicant: Joseph Patrick Lundquist, Santa Monica, CA (US)

(72) Inventor: Joseph Patrick Lundquist, Santa Monica, CA (US)

(21) Appl. No.: 16/105,174

(22) Filed: Aug. 20, 2018

Related U.S. Application Data
(60) Provisional application No. 62/570,958, filed on Oct. 11, 2017.

(51) Int. Cl. A63B 23/18; A61M 16/00

(52) U.S. Cl. CPC A63B 23/18; A61M 16/0006; A61M 2209/088; A61M 2205/0238; A61M 2210/1028; A61M 2205/0205

(57) ABSTRACT

The improved apparatus for semi-occluded vocal tract exercises disclosed herein may comprise a novel hollow apparatus that enables and enhances semi-occluded vocal tract breathing and vocalizing exercises. A preferable embodiment comprises a hollow tube. The tube may be made of metal or another durable material. It may be rigid or flexible in different embodiments. In preferred embodiments, the tube would feature a material that has antimicrobial properties such as the oligodynamic effect found in copper, brass, silver, gold, and several other metals and alloys. In certain embodiments it might be approximately 76.2 mm long, with an inner diameter between 2.3 mm and 3.5 mm. In preferred embodiments, the apparatus would have an inner diameter between 2.3 mm and 3.5 mm. In certain embodiments of the present invention, the tube is incorporated into a wearable item such as a necklace. In other embodiments, it is used as an instrument, or as a vocal warm-up tool.

### TECHNICAL FIELD OF THE INVENTION

The present invention relates in general to vocal exercise devices, and, more specifically, to an improved apparatus for semi-occluded vocal tract exercises.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

The vocal tract is the cavity in human beings where the sound produced in the larynx is filtered. A training method known as semi-occluded vocal tract (SOVT) training exists, which specifically involves moving air through a partially-closed mouth to stretch and balance the vocal folds while placing less impact and stress on them.

Persons who wish to take advantage of SOVT training often do so by using an ordinary drinking straw or even a coffee stirring straw to allow a small portion of air out of their mouth at a time. This is referred to as straw phonation. It has been studied and demonstrated in scientific journals that by partially occluding the vocal tract in this manner, many desirable benefits are gained. Among these are effects that foster healthy phonation, such as increased inertance of the vocal tract, proper alignment of the vocal tract, correction of breathing techniques, and development of phonation muscle memory.

Increased inertance of the vocal tract is the keeping of air in the vocal tract's contained acoustic system by restricting the ease with which air flows out of the vocal tract, in this case by increased back-pressure. The level of this increase has a direct relationship with the length of the straw or tube used, and has an inverse relationship with the inner diameter of the straw or tube used. Increasing inertance in this manner allows the vocal cords to approach each other in a more controlled, balanced, and easy way, increasing vibration efficiency and loudness, and improving acoustic focus.

It is somewhat of an “off-label” use for a drinking straw to blow air out instead of drinking liquid in. While straws often provide the needed resistance to airflow to gain some of these benefits, many people find themselves obtaining their straws from any number of fast food restaurants or cafes whenever the chance arises. There are several problems inherent in this use. The straws in these establishments are designed for one-time use, and continued use is not sanitary. Continued use is often not even possible either because though a fast food straw is rigid, it is not durable and will kink or puncture. These one-time-use straws lead to extra waste that is not good for the environment. Further, these straws are not ideal for the serious vocalist because there is no standardized drinking straw size. A focus of SOVT training is consistency in practice, and inconsistency in straw size prohibits this from the start.

Despite the inconsistent diameters, lengths, and materials of typical drinking straws, there are no drinking straws designed to be small and durable enough as to fit comfortably into pockets and reuse as a SOVT training device over and over. Due to the basic nature of drinking straws, they are often longer than ideal for portability in a small pocket or purse.

There is currently a device, the Doctorvox voice therapy and vocal training device (U.S. Patent Application US20170348504A1) which attempts to address similar voice therapy concerns as does the present invention. However, the Doctorvox's inclusion of goals such as humidification of vocal folds and being an inhalation and spray device produces a result that is less than ideal for regular and ongoing portability and use.

Therefore, a need exists in the field for novel SOVT training devices that are portable, durable, and sanitary for ongoing use. It is to these ends that the present invention has been developed.

### BRIEF SUMMARY OF THE INVENTION

To minimize the limitations in the prior art, and to minimize other limitations that will be apparent upon reading and understanding the present specification, the present invention describes an improved apparatus for semi-occluded vocal tract exercises.

It is an objective of the present invention to provide an improved apparatus for semi-occluded vocal tract exercises that may comprise a hollow tube.

It is another objective of the present invention to provide an improved apparatus for semi-occluded vocal tract exercises that may comprise a resilient material.

It is another objective of the present invention to provide an improved apparatus for semi-occluded vocal tract exercises that may comprise a rigid material.

It is another objective of the present invention to provide an improved apparatus for semi-occluded vocal tract exercises that may comprise a flexible material.

It is another objective of the present invention to provide an improved apparatus for semi-occluded vocal tract exercises that may comprise antimicrobial properties.

It is another objective of the present invention to provide an improved apparatus for semi-occluded vocal tract exercises that may comprise a wearable device.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates a first embodiment of an improved apparatus for semi-occluded vocal tract exercises from a side perspective.

FIG. 2 illustrates a first embodiment of an improved apparatus for semi-occluded vocal tract exercises from a side three-quarters perspective.

FIG. 3 illustrates a first embodiment of an improved apparatus for semi-occluded vocal tract exercises from a top three-quarters perspective.

FIG. 4 illustrates a second embodiment of an improved apparatus for semi-occluded vocal tract exercises from a side perspective.

FIG. 5 illustrates a second embodiment of an improved apparatus for semi-occluded vocal tract exercises from a side three-quarters perspective.

FIG. 6 illustrates a second embodiment of an improved apparatus for semi-occluded vocal tract exercises from a top three-quarters perspective.

FIG. 7 illustrates a second embodiment of an improved apparatus for semi-occluded vocal tract exercises from a rear perspective.

FIG. 8 illustrates a third embodiment of an improved apparatus for semi-occluded vocal tract exercises from a front perspective.

FIG. 9 illustrates a third embodiment of an improved apparatus for semi-occluded vocal tract exercises from a side three-quarters perspective.

FIG. 10 illustrates a third embodiment of an improved apparatus for semi-occluded vocal tract exercises from a rear perspective.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The present invention relates in general to vocal exercise devices, and, more specifically, to an improved apparatus for semi-occluded vocal tract exercises. As contemplated by the present invention, the term “vocal straw” may used to describe the device, as the preferred embodiment is meant to resemble a simple drinking straw, which the invention replaces. The present invention may be made available in any appropriate shape, though, namely oval, rectangular, star-shaped, or other formed shapes.

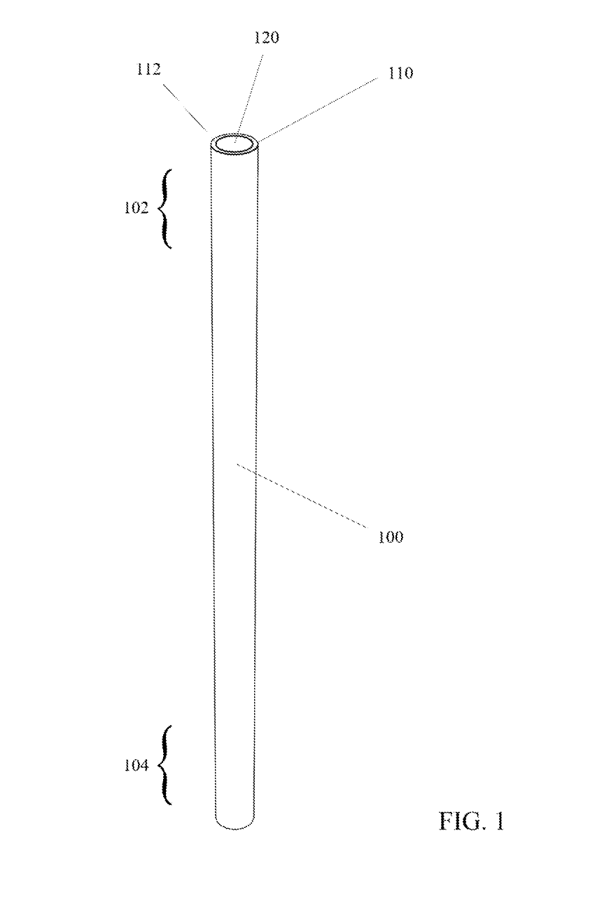

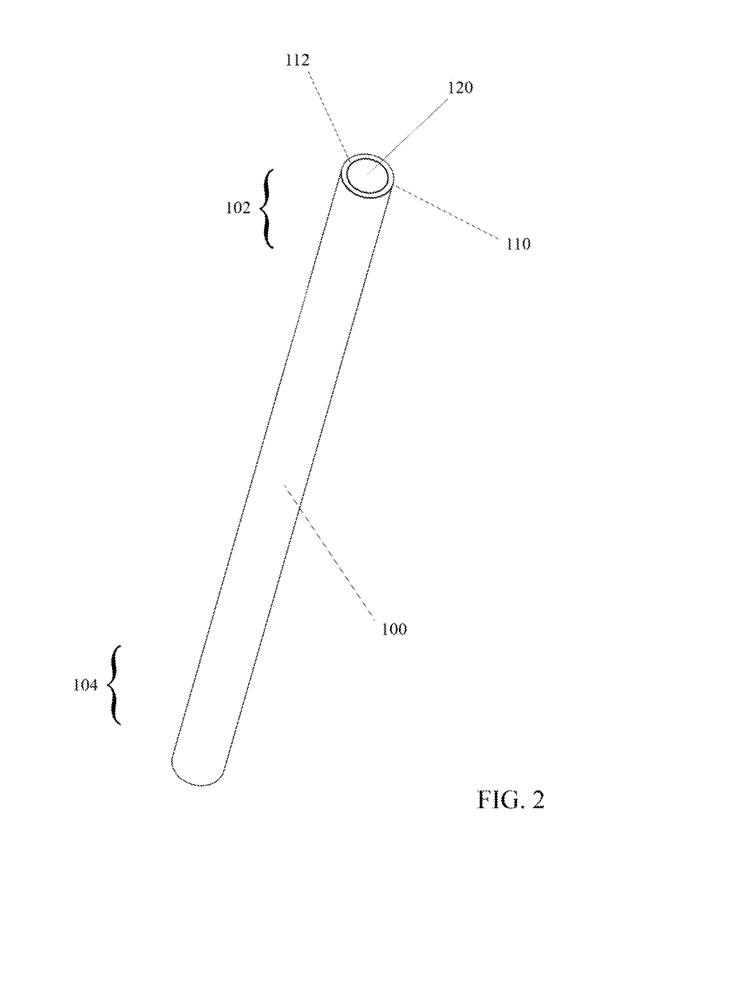

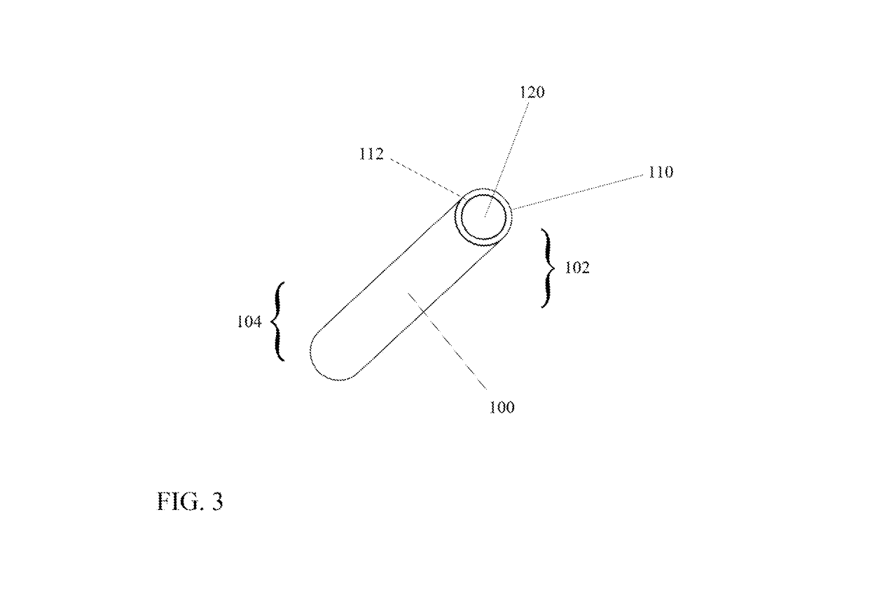

FIGS. 1 through 3 illustrate a first embodiment of an improved apparatus for semi-occluded vocal tract exercises, as contemplated by the present invention. The figures identify a main body 100 having a first end 102 and a second end 104. The main body 100 comprises a substantially tubular shape having an outer diameter 110, an inner diameter 112, and forming a central lumen 120.

The present invention comprises a novel hollow apparatus that enables and enhances semi-occluded vocal tract breathing and vocalizing exercises. A preferable embodiment comprises a hollow tube. The main body may be made of any appropriate material, though preferably comprises a resilient material so as to be durable in order to withstand regular and continued use. By way of example, the tube may comprise a material of construction of copper, aluminum, steel, brass, silver, gold, carbon fiber, fiberglass, or any combination thereof.

In an alternative embodiment the present invention may comprise a flexible material of construction so as to be collapsible or so that it may be rolled up for portability. In certain embodiments, the material of construction either comprises a material having antimicrobial properties or comprises a layering of antimicrobial material or coating. Antimicrobial properties comprise antibacterial, biocidal, microbicidal, anti-fungal, anti-viral, or others. One such antimicrobial property is the oligodynamic effect, which is possessed by copper, brass, silver, gold, and several other metals and alloys.

The incorporation of antimicrobial properties is an important aspect to this invention. Copper and its alloys in particular have exceptional self-sanitizing effects. Silver also has this effect, and is less toxic to humans than copper. Some materials, such as silver in its metallic form, may require the presence of moisture to activate antimicrobial properties.

In preferred embodiments, the apparatus would have an inner diameter between 2 mm and 4 mm. Given that vocal tract inertance is inversely proportional to the inner diameter of a straw, the smaller the diameter, the more difficult it is to move air through it. When a person has a naturally smaller vocal tract relative to the average person, or they have a smaller volume of air that typically is expelled when vocalizing, relative to the average person, they may prefer a larger inner diameter for a vocal straw. This is the opposite for a person with larger than average vocal tract or volume of air expelled when vocalizing, which person may prefer the greater resistance provided by a smaller inner diameter.

In certain embodiments, the vocal straw might be approximately 76.2 mm long, with an inner diameter between 2.3 mm and 3.5 mm because this is a small enough opening that it provides needed resistance for those with relatively strong exhaling ability to gain the desired effects of SOVT exercises. At the same time, it is large enough so as to not prohibit those with relatively less exhaling ability from effectively using the straw for SOVT exercises. The 76.2 mm length is an excellent size for portability. Furthermore, because some materials including silver require moisture for active antimicrobial effect, the anaerobic nature of narrow tubing is beneficial to the antimicrobial function of the invention, allowing moisture from saliva and condensation to remain in the tube.

In certain embodiments the overall structure of the device may comprise a shape that is non-tubular, though a tubular air flow channel may be enveloped within the shape itself. In such embodiments the shape may comprise an air inlet and exit substantially opposed to one another. By way of example, if the main body is shaped as a sphere or disc, the air inlet may be at one end and the air exit may be located 180 degrees opposite to the air inlet. An air tube may connect the air inlet and exit through the main body. In another example, if the main body is shaped as a star the air inlet may be at one point while the air exit may be at any one or more other points. The air channel connecting the inlet and exits may or may not be a straight channel, and may comprise various diameters that may be selectively blocked so that a user may manually adjust the resistance to air flow.

In all embodiments it is contemplated that the device may have an adjustable length or an adjustable air channel diameter or resistance. A single user may have need for varying air flow resistances based on individual training or need, and so a single device may be able to suit these varying needs for the individual user. In the alternative, multiple users may have differing air flow resistance needs, and so a single device design may be usable by users of varying needs.

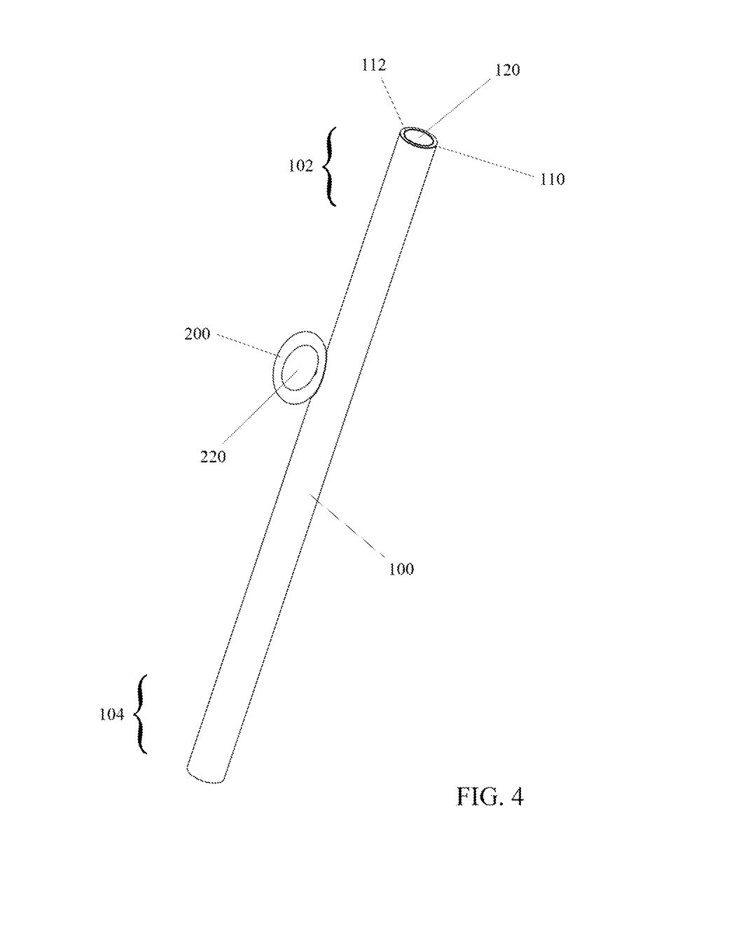

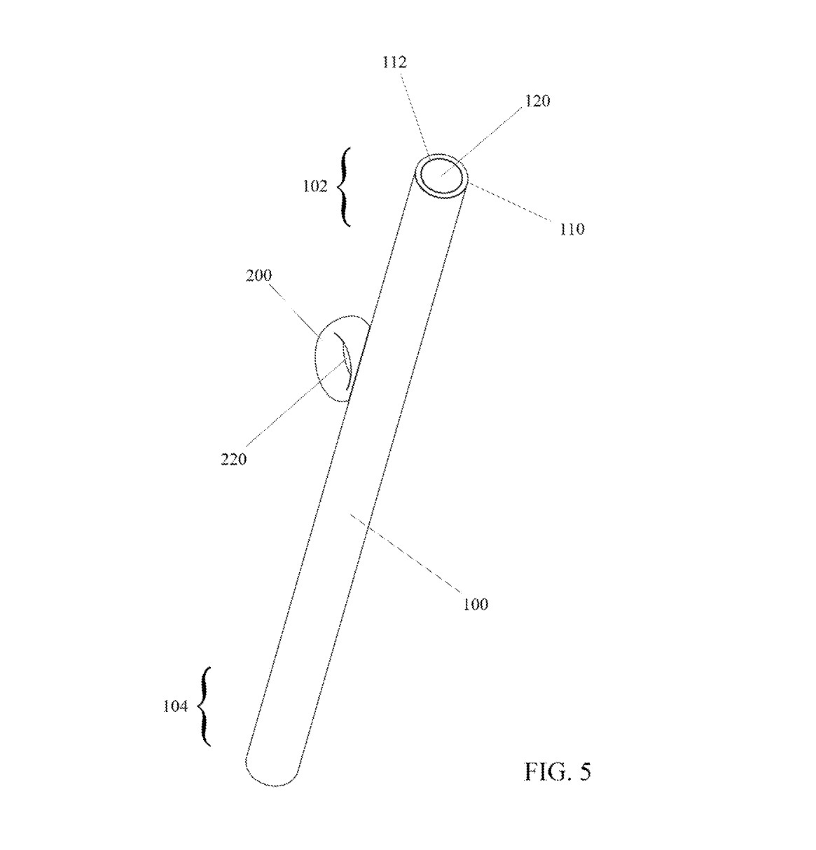

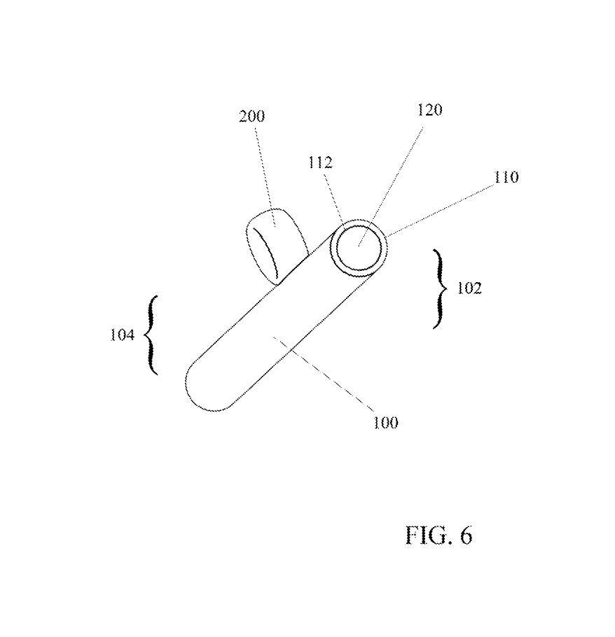

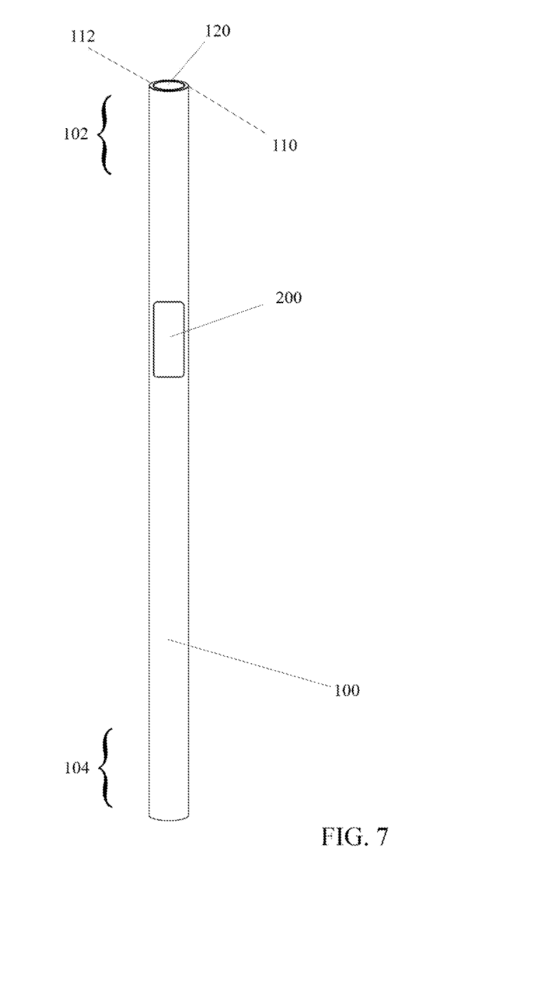

FIGS. 4 through 7 illustrate a second embodiment of an improved apparatus for semi-occluded vocal tract exercises, as contemplated by the present invention. The figures identify a main body 100 having a first end 102 and a second end 104. The main body 100 comprises a substantially tubular shape having an outer diameter 110, an inner diameter 112, and forming a central lumen 120. Further illustrated is a means for suspending 200 having a central opening 220.

In certain embodiments of the present invention, the tube is incorporated into a wearable item such as a necklace. A bail may be soldered onto the vocal straw, or perhaps the entire apparatus is cast or 3D-printed, or is constructed by some other appropriate means. In one embodiment, the means for suspending 200 would enable the vocal straw to be suspended on a necklace by passing it through the central opening 220. In another embodiment with perhaps a different size means for suspending 200, the vocal straw could be held easily by or worn on a finger or fingers. The loop could also be rotated on its axis or duplicated in certain embodiments.

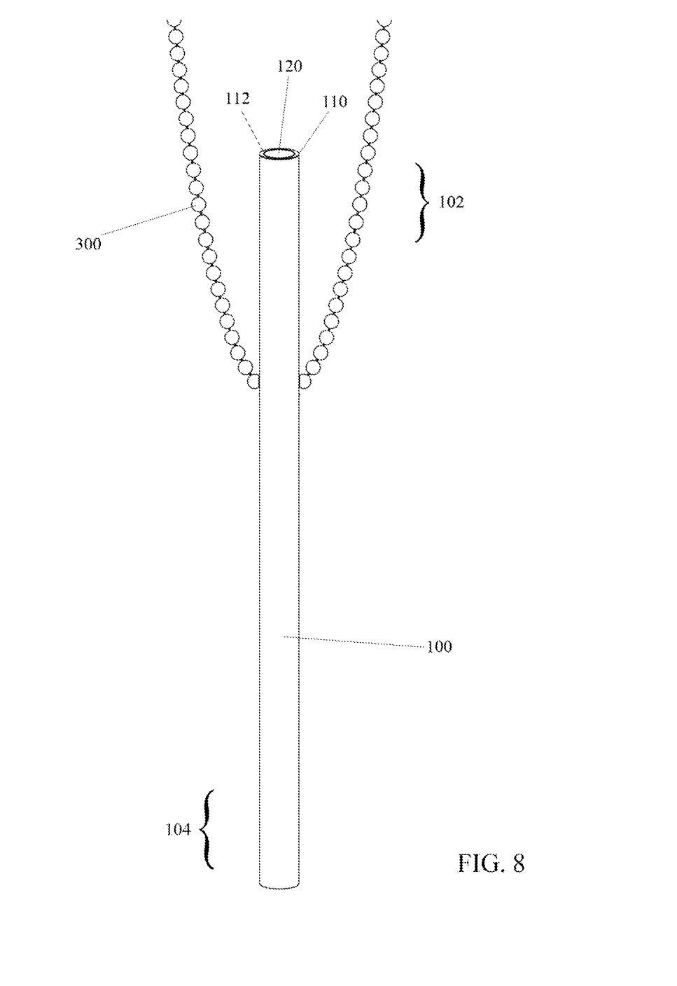

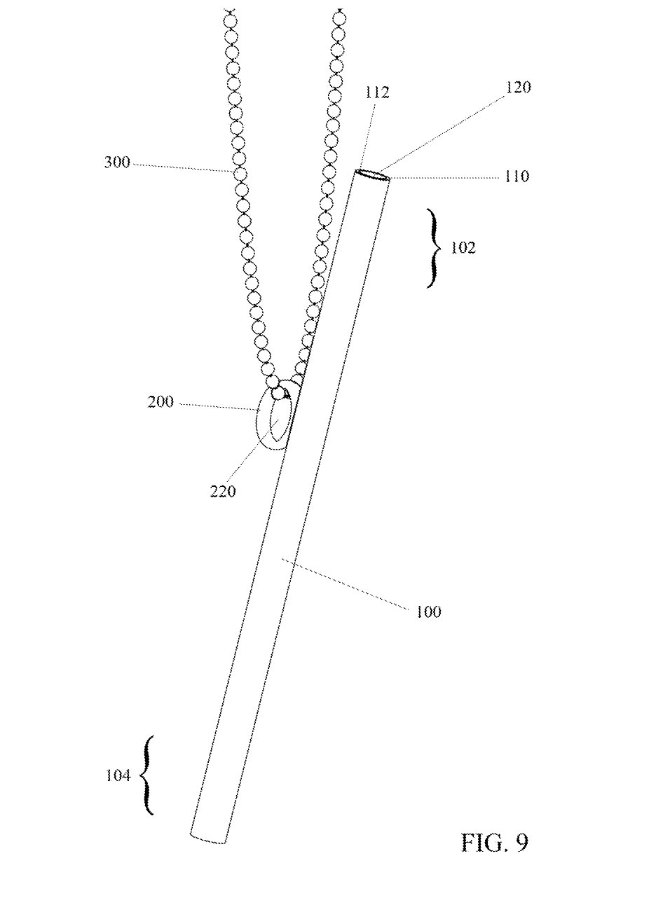

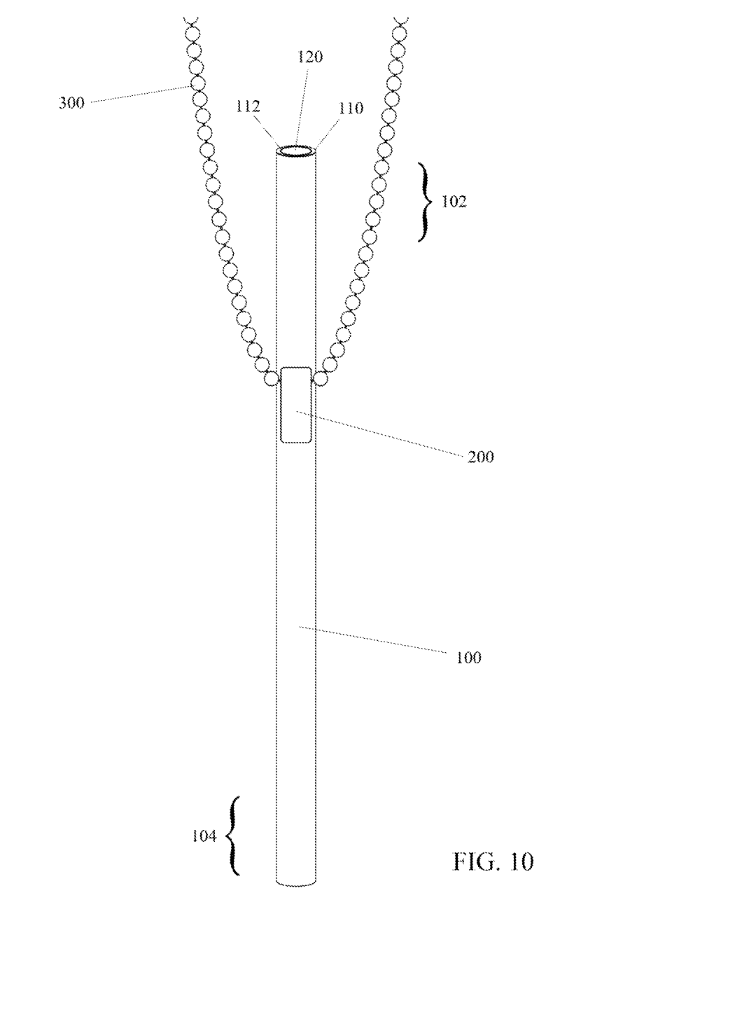

FIGS. 8 through 10 illustrate a third embodiment of an improved apparatus for semi-occluded vocal tract exercises, as contemplated by the present invention. The figures identify a main body 100 having a first end 102 and a second end 104. The main body 100 comprises a substantially tubular shape having an outer diameter 110, an inner diameter 112, and forming a central lumen 120. Further illustrated are a means for suspending 200, having a central opening 220, and a means for wearing 300.

In a preferred embodiment the means for wearing 300 is a necklace, chain, rope, or any other suitable means for engaging a user. In some embodiments, the vocal straw is used as an instrument that facilitates a distinct timbre in the voice. In some embodiments it may be used as a vocal warm-up tool, wherein the means for suspension 200 may engage a user's finger. The vocal straw may be used for acute vocal therapy or for ongoing vocal health maintenance. The vocal straw may also be used as a noise-dampening device, which is particularly beneficial in environments where a user is in close proximity to others, such as in a city or apartment. In certain embodiments, the invention may be used without vocalization. An example of this would be a reed instrumentalist using the invention for breathing exercises to train in economy of breath.

In some embodiments, the means for suspending 200 may possess one or more inner concentric loops that facilitate the spinning of the vocal straw around the axis of the concentric loops. In another embodiment, magnets, weights, bearings or lubricants may be incorporated to facilitate such spinning.

In a preferred embodiment the device may further incorporate a means for attachment. Such a means for attachment may comprise a clip, a clasp, a magnet, or any other appropriate means. A clip or a clasp, for example, may provide the ability to attach the vocal straw to a piece of clothing, a piece of paper, or other items. The use of a magnet, or the use of a magnetic material of construction, may allow the device to attach magnetically to another item for easy storage or for other purposes.

In certain embodiments, the apparatus may be molded in such a way as to be easily held. It may also be incorporated into a larger apparatus which features an ergonomic design.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims. Note with respect to the materials of construction, it is not desired nor intended to thereby unnecessarily limit the present invention by reason of such disclosure.

I claim:

1. An improved apparatus for semi-occluded vocal tract exercises, comprising:
a main body;
an air channel; and
an antimicrobial layer;
wherein said air channel comprises a hollow tube having an outer diameter, an inner diameter, and a length;
wherein said air channel is housed within said main body so as to create an air inlet at a first edge of said main body and an air exit at a second edge of said main body; and
wherein said main body comprises a resilient material of construction.

2. The invention of claim 1,
wherein said main body comprises a geometric shape.

3. The invention of claim 2,
wherein said air channel further comprises a means for adjustment.

4. The invention of claim 3,
wherein said antimicrobial layer comprises an oligodynamic effect of said material of construction.

5. The invention of claim 4,
wherein said antimicrobial layer further comprises an additional layer covering said material of construction.

6. An improved apparatus for semi-occluded vocal tract exercises, comprising:
a main body;
an air channel;
a means for suspending; and
an antimicrobial layer;
wherein said air channel comprises a hollow tube having an outer diameter, an inner diameter, and a length;
wherein said air channel is housed within said main body so as to create an air inlet at a first edge of said main body and an air exit at a second edge of said main body;
wherein said means for suspending is attached to an outer surface of said main body; and
wherein said main body comprises a resilient material of construction.

7. The invention of claim 6,
wherein said main body comprises a geometric shape.

8. The invention of claim 7,
wherein said air channel further comprises a means for adjustment.

9. The invention of claim 8,
wherein said means for suspending comprises a loop.

10. The invention of claim 9,
wherein said antimicrobial layer comprises an oligodynamic effect of said material of construction.

11. The invention of claim 10,
wherein said antimicrobial layer further comprises an additional layer covering said material of construction.

12. An improved apparatus for semi-occluded vocal tract exercises, comprising:
a main body;
an air channel;
a means for suspending;
a means for wearing; and
an antimicrobial layer;
wherein said air channel comprises a hollow tube having an outer diameter, an inner diameter, and a length;
wherein said air channel is housed within said main body so as to create an air inlet at a first edge of said main body and an air exit at a second edge of said main body;
wherein said means for suspending is attached to an outer surface of said main body; and
wherein said main body comprises a resilient material of construction.

13. The invention of claim 12,
wherein said main body comprises a geometric shape.

14. The invention of claim 13,
wherein said air channel further comprises a means for adjustment.

15. The invention of claim 14,
wherein said means for suspending comprises a loop.

16. The invention of claim 15,
wherein said antimicrobial layer comprises an oligodynamic effect of said material of construction.

17. The invention of claim 16,
wherein said antimicrobial layer further comprises an additional layer covering said material of construction.
